(19) United States
(12) Patent Application Publication
(10) Pub. No.: US 2024/0293644 A1
(43) Pub. Date: Sep. 5, 2024

(54) SUBCUTANEOUS TUNNELING CATHETER FOR PERITONEAL ACCESS

(71) Applicants: Patrick MCGLYNN, Palo Alto, CA (US); Fabio KOMLOS, Los Altos, CA (US)

(72) Inventors: Patrick MCGLYNN, Palo Alto, CA (US); Fabio KOMLOS, Los Altos, CA (US)

(21) Appl. No.: 18/429,129

(22) Filed: Jan. 31, 2024

Related U.S. Application Data
(63) Continuation of application No. PCT/US2022/039195, filed on Aug. 2, 2022.
(60) Provisional application No. 63/229,020, filed on Aug. 3, 2021.

(51) Int. Cl. A61M 25/01; A61M 25/00; A61M 25/02

(52) U.S. Cl. CPC A61M 25/0194; A61M 25/0053; A61M 2025/0213; A61M 2205/0205

(57) ABSTRACT

Provided herein are systems, devices, and methods for facilitating the performance of peritoneal dialysis. A long-term use sheath with cuffs to secure to skin and the rectus muscle is provided. Through this long-term use sheath, a peritoneal dialysis catheter may be inserted into the peritoneum and used for dialysis, and when appropriate such as when catheter malfunction risk or injury risk is apparent, this catheter may be exchanged through the sheath for a new catheter.

### CROSS-REFERENCE

This application is a continuation of International Application No. PCT/US2022/039195, filed Aug. 2, 2022, which claims the benefit of U.S. Provisional Patent Application No. 63/229,020, filed Aug. 3, 2021, which is incorporated herein by reference.

### BACKGROUND

Chronic peritoneal dialysis (PD) is often performed by a catheter configured for unrestricted inflow and outflow of a dialysate solution, which is circulated through the catheter placed inside the peritoneal cavity of a patient. As typically used in current practice, catheter malfunctions due to factors such as catheter migration, kinking, fibrin deposition, omental wrapping, obstruction secondary to intraperitoneal adhesions, and the like often reduce the efficacy of dialysis treatments. There are therefore needs for improved peritoneal dialysis catheters and methods of use.

The following references may be of interest: U.S. Pat. Nos. 3,402,710, 3,505,988, 3,633,585, 4,184,497, 4,278,092, 4,321,914, 4,469,835, 4,412,834, 4,475,900, 4,498,902, 4,559,033, 4,687,471, 4,935,004, 5,057,075, 5,098,413, and 5,090,954; PCT Patent Publication No. WO1983002060A1; European Patent No. EP0081724B1; and Chinese Utility Patent No. CN214027U.

### SUMMARY

The present disclosure relates to medical systems, devices, and methods, in particular for peritoneal dialysis. Aspects of the present disclosure provide a semi-permanent, long-term use tunneled sheath for access from the skin to the peritoneum of a subject, into which a peritoneal dialysis (PD) catheter may be advanced for use in a PD or other procedure and then removed and exchanged for subsequent PD or other procedures.

Provided herein is a method of facilitating peritoneal dialysis on a subject, the method comprising: advancing a long-term use sheath through a puncture in skin of a subject and into a peritoneal cavity of the subject; securing a first cuff of the long-term use sheath to a rectus muscle of the subject; and advancing a first peritoneal catheter through the long-term use sheath and into the peritoneum.

In some embodiments, the method further comprises performing peritoneal dialysis through the first peritoneal catheter advanced through the long-term use sheath. In some embodiments, the method further comprises exchanging the first peritoneal dialysis catheter advanced through the long-term use sheath with a second peritoneal dialysis catheter. In some embodiments, exchanging the first peritoneal dialysis catheter with the second peritoneal dialysis catheter comprises removing the first peritoneal dialysis catheter from the long-term use sheath and advancing the second peritoneal catheter through the long-term use sheath and into the peritoneal cavity. In some embodiments, the method further comprises performing peritoneal dialysis through the second peritoneal catheter advanced through the long-term use sheath. In some embodiments, the method further comprises securing a second cuff to the skin of the subject adjacent the puncture. In some embodiments, advancing the long-term use sheath through the puncture in the skin of the subject and into the peritoneal cavity of the subject comprises: (i) penetrating a needle through the skin, subcutaneous tissue under the skin, and the rectus muscle of the subject and into the peritoneum of the subject; (ii) advancing a first guidewire through the needle; (iii) exchanging the first guidewire in the needle with a micropuncture sheath; (iv) advancing a second guidewire into the micropuncture sheath; (v) advancing the second guidewire into a pelvis of the subject; (vi) advancing a semi-permanent sheath over the second guidewire and into the pelvis; and (vii) advancing the long-term use sheath into the semi-permanent sheath. In some embodiments, the method further comprises one or more of cleaning, sterilizing, or draping the subject. In some embodiments, the method further comprises removing the tear-away sheath after the long-term use sheath is advanced into the semi-permanent sheath. In some embodiments, the semi-permanent sheath comprises a tear-away sheath. In some embodiments, the first guidewire comprises a micropuncture guidewire. In some embodiments, the second guidewire comprises a stiff guidewire. In some embodiments, the method further comprises injecting a contrast agent through the needle to confirm that the needle is within the peritoneum, that the needle has not punctured the bowel, or both. In some embodiments, the needle is advanced into the peritoneum at an entry angle of about 45 degrees. In some embodiments, the method further comprises allowing the second guidewire to form a loop while the second guidewire is advanced into the micropuncture sheath. In some embodiments, the method further comprises inflating an intra-peritoneal disc of the subject after the long-term sheath is advanced into the pelvis. In some embodiments, the method is performed in an operating room or an interventional suite. In some embodiments, the long-term use sheath is advanced through the puncture in the skin of the subject and into the peritoneal cavity of the subject under image guidance. In some embodiments, the image guidance comprises fluoroscopy, computed tomography, magnetic resonance imaging, ultrasound, or any combination thereof. In some embodiments, the method further comprises applying a local anesthetic to the subject. In some embodiments, the method further comprises dilating the rectus muscle. In some embodiments, the method further comprises coupling the catheter to a proximal hub. In some embodiments, the method further comprises cutting the long-term use sheath to a desired length. In some embodiments, the method further comprises sealing the long-term use sheath after the long-term use sheath has been advanced into the peritoneum. In some embodiments, the first cuff is made at least partially from Dacron. In some embodiments, at least a portion of the long-term use sheath is perforated. In some embodiments, the method further comprises bending at least a distal portion of the long-term use sheath toward a pelvis. In some embodiments, the method further comprises sterilizing the long-term use catheter. In some embodiments, the method further comprises delivering light to the long-term use catheter via an illuminating trocar. In some embodiments, the delivered light comprises ultraviolet (UV) light and the delivered UV light sterilizes the long-term use catheter. In some embodiments, the long-term use sheath is advanced into the peritoneal cavity of the subject through a trocar advanced through the skin of the subject.

Another aspect provided herein is a long-term use sheath for facilitating peritoneal dialysis, the long-term use sheath comprising: a sheath body having an inner lumen, wherein the lumen is configured to accommodate the advancement of a first peritoneal dialysis therethrough and exchange of the first peritoneal dialysis with a second peritoneal dialysis catheter; a first cuff coupled to the sheath body and adapted to secure to a rectus muscle of the subject; and a second cuff coupled to the sheath body and adapted to secure to skin of the subject.

In some embodiments, at least a portion of the long-term use sheath is impregnated with one or more of an anti-microbial agent, an anti-bacterial agent, such as copper or silver. In some embodiments, at least a portion of the long-term use sheath is pliable. In some embodiments, at least a portion of the long-term use sheath is rigid. In some embodiments, at least a portion of the long-term use sheath has a rigid bend. In some embodiments, at least a portion of the long-term use catheter is weighted. In some embodiments, at least a portion of the long-term use catheter comprises a leak sensor.

Another aspect provided herein is a kit for implanting a long-term use sheath for conducting peritoneal dialysis, the kit comprising: (i) a needle to be penetrated across skin, subcutaneous tissue under the skin, and a rectus muscle of the subject and into the peritoneum of the subject; (ii) a first guidewire for advancement through the needle; (iii) a micropuncture sheath; (iv) a stiff guidewire for advancement into the micropuncture sheath; (v) a tear-away sheath; and (vi) the long-term use sheath.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an illustration of a peritoneal cavity and surrounding anatomy of a subject in which an exemplary method of facilitating peritoneal dialysis may be performed, in accordance with embodiments described herein; and

FIGS. 2A-2F shows side, section views of an exemplary method of facilitating peritoneal dialysis performed on a subject, in accordance with embodiments described herein.

FIGS. 3 and 4 show a side, section views of a long-term use sheath implanted in a subject to allow access to the peritoneal cavity of the subject with exchangeable peritoneal dialysis catheters, in accordance with embodiments herein.

### DETAILED DESCRIPTION

Leaking and other malfunctions associated with migration of current PD catheters include kinking, fibrin deposition, omental wrapping, obstructions, or any combination thereof. Such malfunctions cause inadequate dialysis, dialysis insecurity (e.g., uncertainty of where and how next dialysis treatment will occur) and may require a temporary or permanent transition from PD to hemodialysis. Further, current catheters may be dislodged or damaged by the peristaltic movement of the bowel and can be difficult to drain. Such malfunctions induce pain and missed dialysis treatments.

As such, provided herein is are methods and devices for facilitating peritoneal dialysis. In some embodiments, the methods and devices herein provide a tunneled sheath for access to the peritoneum, into which a further catheter can be advanced through the existing tunnel of the sheath and into the peritoneum.

In some embodiments, the devices and methods herein are configured for immediate placement, use, and exchange without dialysis disruption. In some embodiments, the devices and methods herein improve exchangeability of a peritoneal dialysis catheter inside of an interventional suite. In some embodiments, the ability of the catheters herein to be reliably located in the true pelvis and/or peritoneum, without kinking, enables their immediate placement. Further, the catheters herein are configured to reduce infection and subcutaneous leakage by minimizing exposure in the abdomen. Such reduced exposure prevents the development of adhesions, omental wrapping, fibrin deposition, obstructions, or any combination thereof to ensure regular and adequate dialysis and prevents the need for the subject to temporarily or permanently transfer to hemodialysis. Additionally, the methods and devices herein are secured within the anatomic structure or lumen to prevent dislodgement and/or damage by the peristaltic movement of the bowel.

### Method of Facilitating Peritoneal Dialysis on a Subject

Provided herein is a method of facilitating peritoneal dialysis on the subject, the method comprising positioning a long-term use sheath to provide access to the peritoneum from the skin, for example, at the abdomen of the subject, the long-term use sheath facilitating the advancement of peritoneal catheters to access the peritoneum and facilitating exchange of such catheters. The long-term-term use sheath may be positioned to the desired location using a method comprising the steps of: (i) penetrating a needle through the skin and subcutaneous tissue under the skin, and the rectus muscle of the subject and into the peritoneum of the subject; (ii) advancing a first guidewire through the needle; (iii) exchanging the needle with a micropuncture sheath over the first guidewire; (iv) advancing the micropuncture sheath into the peritoneum of the subject; (v) removing the first guidewire; (vi) advancing a second guidewire into the micropuncture sheath; (vi) advancing the second guidewire into the peritoneum of the subject; (vii) removing the micropuncture sheath; (viii) advancing a semi-permanent sheath over the second guidewire and into the peritoneum; (ix) advancing the long-term use sheath into the semi-permanent sheath; and (x) positioning cuffs of the long-term use sheath to subcutaneous tissue and/or the rectus muscle to fixate the long-term use sheath.

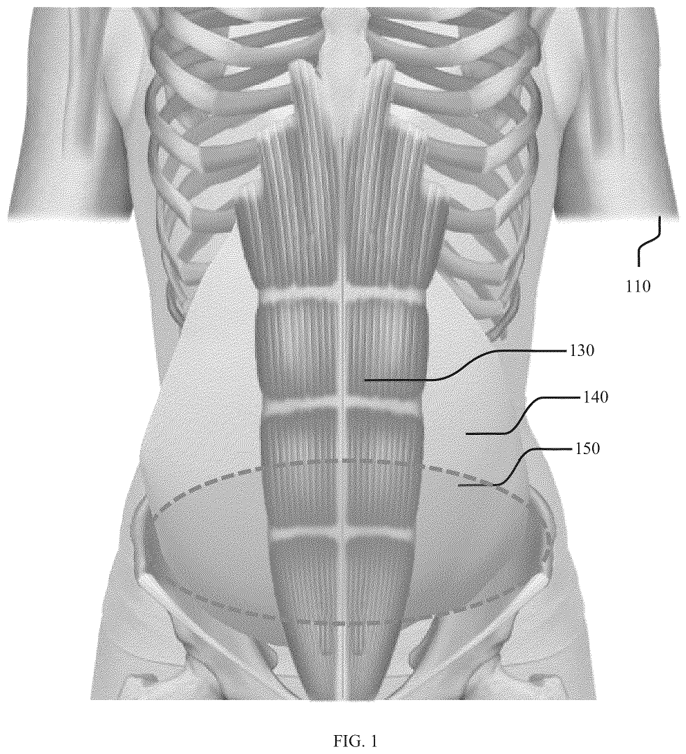

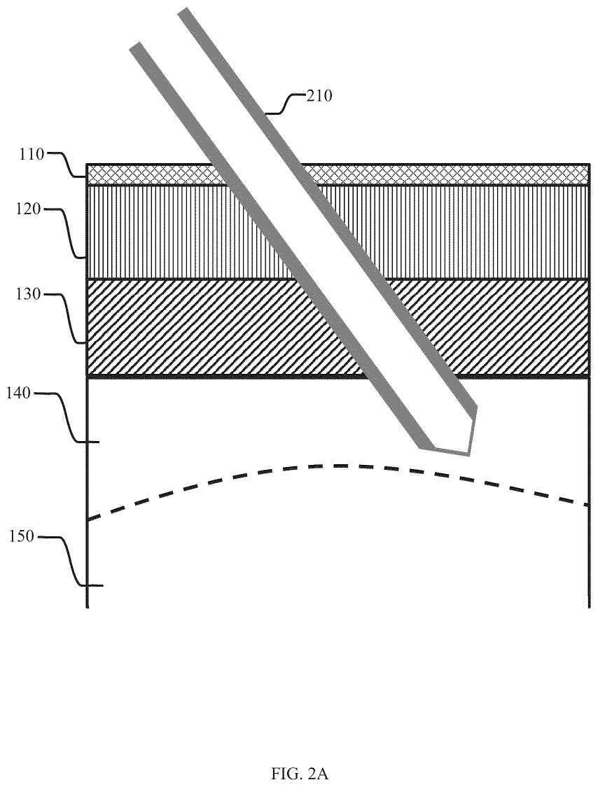

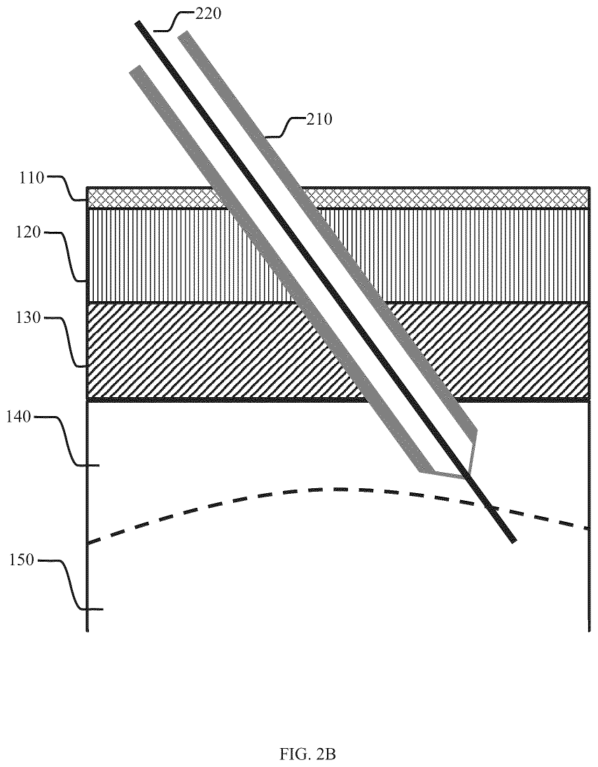

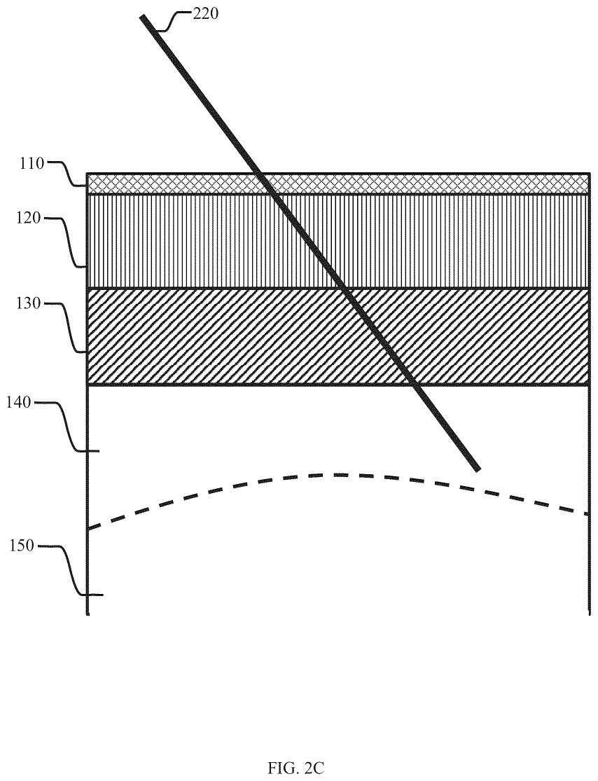

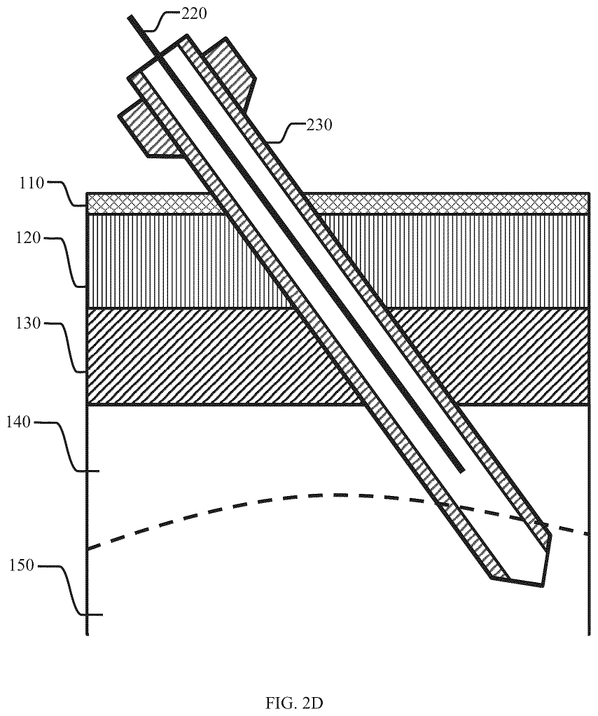

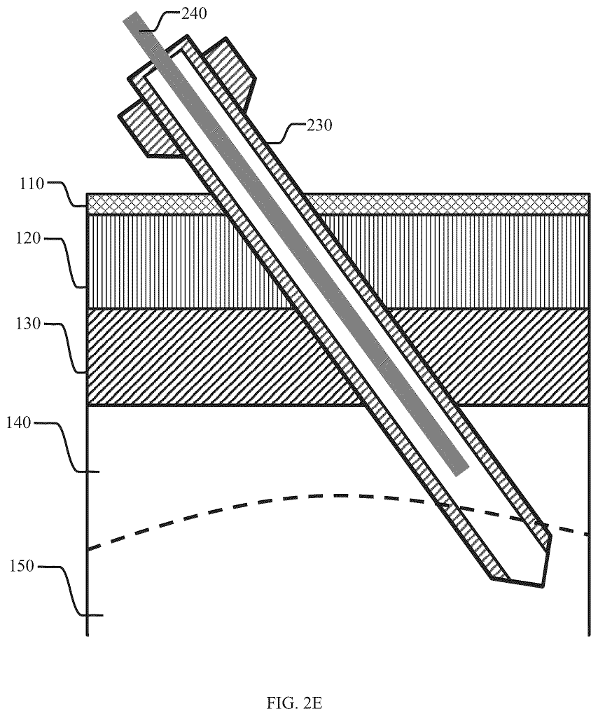

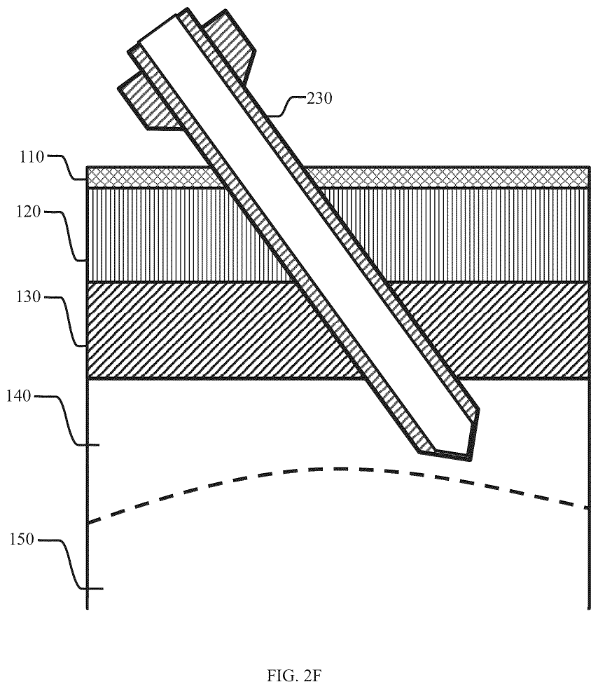

FIGS. 1-2F show the anatomy of a subject. The applicable anatomy of the subject includes their skin 110, the rectus muscle 130, the peritoneum or peritoneal cavity 140, and the true pelvis 150. Further, the true pelvis 150 (or lesser pelvis) encloses the peritoneal space between the bladder and rectum or uterus.

FIGS. 2A-2F show an exemplary method of facilitating peritoneal dialysis on the subject, in particular, for placing a long-term use sheath in position to facilitate the use of PD catheters and their exchange. In some embodiments, the method comprises: (i) penetrating a needle 210 through the skin 110, the subcutaneous tissue 120, and the rectus muscle 130 of the subject, per FIG. 2A; (ii) advancing a first guidewire 220 through the needle 210 and into the peritoneum 140 of the subject, per FIG. 2B; (iii) removing the needle 210, per FIG. 2C; (iv) advancing a micro-puncture sheath 230 over the first guidewire 220, per FIG. 2D; and (v) replacing the first guidewire 220 with a second guidewire 240, per FIG. 2E.

In some embodiments, per FIG. 2F, the method further comprises removing the second guidewire 240. In some embodiments, the method further comprises tunneling the micro-puncture sheath 230 over the second guidewire 240. In some embodiments, the method further comprises removing the second guidewire 240. In some embodiments, the micro-puncture sheath 230 is advanced through an anatomic structure or lumen of the subject such as to the true pelvis 150 of the subject. In some embodiments, the method further comprises advancing the second guidewire 240 to the true pelvis of the subject and advancing the micro-puncture sheath 230 to the true pelvis, before removing the second guidewire 240.

In some embodiments, the method further comprises confirming that the needle 210 is within the true pelvis 150 of the subject or has otherwise access to the peritoneum in the steps of FIGS. 2A-2B. In some embodiments, confirming that the needle 210 is within the true pelvis 150 of the subject comprises: using image guidance; using ultrasound guidance; using fluoroscopy; injecting a contrast agent into the true pelvis 150; measuring a flow resistance covering at least a portion of the needle 210 with a radio-opaque marker; or any combination thereof. In some embodiments, confirming that the needle 210 is within the true pelvis 150 ensures proper needle placement, especially if the kidneys are atrophic. In some embodiments, the first guidewire 220, the micro-puncture sheath 230, or both has a distal transducer to enable ultrasound guidance. In some embodiments, the flow resistance is measured by an injector.

In some embodiments, image guidance comprises performing an ultrasound, X-ray, fluoroscopy, or other imaging while injecting the needle 210 or micro-puncture sheath 230, wherein contrast between the needle 210 or micro-puncture sheath 230 and the subject's organs enables proper placement. In some embodiments, the contrast agent is a fluorographic dye that enhances contrast between the needle 210 or micro-puncture sheath 230 and the subject's organs under X-ray or fluoroscopy. Alternatively or in combination, the contrast agent is an echogenic agent that enhances contrast between the needle 210 or micro-puncture sheath 230 and the subject's organs under ultrasound. In some embodiments, the radio-opaque marker is a dye that enables contrast between the needle 210 or micro-puncture sheath 230 and the subject's organs under radio waves.

In some embodiments, the method further comprises confirming that the micro-puncture 230 is within the true pelvis 150 of the subject or has otherwise access to the peritoneum of the subject in the steps of FIGS. 2D-2F. In some embodiments, confirming that the micro-puncture sheath 230 is within the peritoneum of the subject comprises: using image guidance; using ultrasound guidance using fluoroscopy; injecting a contrast agent into the true pelvis 150; measuring a flow resistance performing a pyelography; covering at least a portion of the catheter 230 with a radio-opaque marker; or any combination thereof.

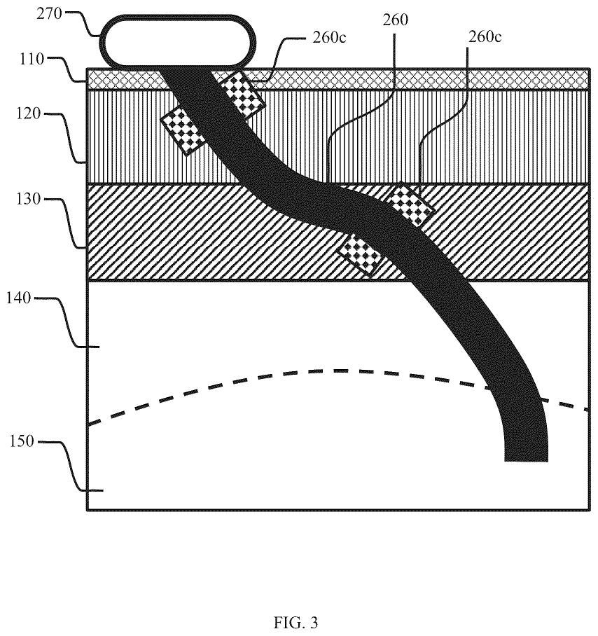

In some embodiments, the method further comprises replacing the first guidewire 220 with floppy tip wire. In some embodiments, the floppy tip wire prevents internal damage that the sharp tipped first guidewire 220 may induce during use. In some embodiments, the method further comprises advancing a long-term use sheath 260 through the micro-puncture 230 as shown in FIG. 3. The long-term use sheath 260 may be advanced from the skin to the true pelvis 150 of the subject to access the peritoneum. Once in position, the micro-puncture sheath 230 may be retracted over the sheath 260. The sheath 260 may comprise cuffs 260c positioned on the sheath 260 such that the cuffs 260c are positioned just under the skin 110 of the subject and at the rectus muscle of the subject when the micro-puncture sheath 230 is retracted, thereby securing the sheath 260. In some embodiments, the micro-puncture sheath 230 is exchanged over the second guide wire 240 with a tear-away sheath which may better facilitate the advancement of the sheath 260 into the anatomy better than a micro-puncture sheath 230. For instance, the tear-away sheath may have a larger lumen and/or a more expandable lumen. Once the sheath 260 is in place, the tear-away sheath may be tom away leaving the cuffs 260c interfacing the skin and the rectus muscle and securing the long term use sheath 260.

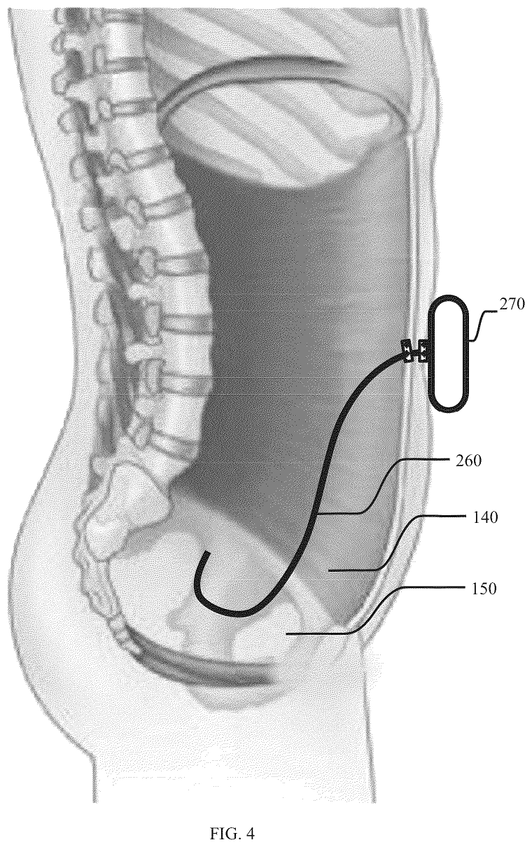

In some embodiments, per FIG. 4, the sheath 260 further comprises an expandable cuff 270 which may be positioned and expanded over the skin 110 to further secure the sheath 260. In some embodiments, the cuffs 260c secure the expandable cuff 270 to the skin 110, the subcutaneous tissue 120, the rectus muscle 130, or any combination thereof, of the subject. In some embodiments, the cuffs 260c, the expandable cuff 270, or both secure the sheath 260 and maintain its position through the peritoneum 140 and into the true pelvis 150.

The catheter(s) for actual peritoneal dialysis may be advanced through the sheath 260 to access the peritoneum. The sheath 260 can facilitate the exchange of a first peritoneal dialysis catheter with a second peritoneal dialysis catheter, such as when the first peritoneal dialysis catheter may be older and needs replacement. Upon long-term implantation, fibrin sheaths may form on the surface of the peritoneal dialysis catheter and/or on the inner surface of the sheath 260. The fibrin sheath may be disrupted on the exchange with the second peritoneal dialysis catheter.

In some embodiments, the needle 210 has a size of 15 gauge to 25 gauge. In some embodiments, the needle 210 has a size of 15, 16, 17, 18, 19, 20, 21, 22, 23, 24 or 25 gauge. In some embodiments, the needle 210 has a size of at least 15, 16, 17, 18, 19, 20, 21, 22, 23, or 24 gauge. In some embodiments, the needle 210 has a size of at most 16, 17, 18, 19, 20, 21, 22, 23, 24 or 25 gauge. In some embodiments, at least a portion of a tip of the needle 210 is straight. In some embodiments, at least a portion of a tip of the needle 210 is curved. In some embodiments, the needle 210 is tunneled. In some embodiments, the needle 210 reduces infections during the procedure. In some embodiments, a distal portion of the needle 210 comprises a plurality of openings to facilitate exchange of fluid therethrough.

In some embodiments, a distal tip of the first guidewire 220 comprises sharp tip for creating a micro-puncture. In some embodiments, the sharp tip punctures the peritoneal membrane.

In some embodiments, the needle 210, the first guidewire 220, the micro-puncture sheath 230, the second guidewire 240, or any combination thereof are formed of metal, plastic, wood, carbon fiber, glass, or any combination thereof. In some embodiments, a flexural modulus (or stiffness) of the micro-puncture sheath 230 is greater than a flexural modulus of the first guidewire 220, the second guidewire 240, or both. In some embodiments, a flexural modulus (or stiffness) of the micro-puncture sheath 230 is greater than a flexural modulus of the first guidewire 220, the second guidewire 240, or both by about 10 GPa to about 80 GPa. In some embodiments, a flexural modulus of the micro-puncture sheath 230 is greater than a flexural modulus of the first guidewire 220, the second guidewire 240, or both by about 10, 20, 30, 40, 50, 60, 70, or 80 GPa. In some embodiments, a flexural modulus of the micro-puncture sheath 230 is greater than a flexural modulus of the first guidewire 220, the second guidewire 240, or both by at least about 10, 20, 30, 40, 50, 60, or 70 GPa. In some embodiments, a flexural modulus of the micro-puncture sheath 230 is greater than a flexural modulus of the first guidewire 220, the second guidewire 240, or both by at most about 20, 30, 40, 50, 60, 70, or about 80 GPa. In some embodiments, the lesser rigidity of the first guidewire 220, the second guidewire 240, or both enables their exit from the catheter 230 at an angle of about 45 degrees.

In some embodiments, the needle 210 is advanced into the peritoneum 150 at an entry angle of about 25 degrees to about 65 degrees. In some embodiments, the needle 210 is advanced into the peritoneum 150 at an entry angle of about 25, 30, 35, 40, 45, 50, 55, 60, or 65 degrees. In some embodiments, the needle 210 is advanced into the peritoneum 150 at an entry angle of at most about 25, 30, 35, 40, 45, 50, 55, or 60 degrees. In some embodiments, the needle 210 is advanced into the peritoneum 150 at an entry angle of about 30, 35, 40, 45, 50, 55, 60, or 65 degrees. In some embodiments, the first guidewire 220 is advanced from within the micro-puncture sheath 230 at an angle with respect to the peritoneal membrane of about 25 degrees to about 65 degrees. In some embodiments, the first guidewire 220 is advanced from within the micro-puncture sheath 230 at an angle with respect to the peritoneal membrane of about 25, 30, 35, 40, 45, 50, 55, 60, or 65 degrees. In some embodiments, the first guidewire 220 is advanced from within the micro-puncture sheath 230 at an angle with respect to the peritoneal membrane of at most about 25, 30, 35, 40, 45, 50, 55, or 60 degrees. In some embodiments, the first guidewire 220 is advanced from within the micro-puncture sheath 230 at an angle with respect to the peritoneal membrane of about 30, 35, 40, 45, 50, 55, 60, or 65 degrees.

### Placement of Long-Term Use Sheath for Facilitating Peritoneal Dialysis Using Laparoscopic Approach

Also provided herein are further methods of placing the long-term use sheath for facilitating peritoneal dialysis using other approaches, for instance, a laparoscopic approach. In some embodiments, the method comprises one or more steps of: placing the patient in a supine position and administering a general anesthesia, forming a pneumoperitoneum with an open procedure; forming a 2-3 cm subumbilical incision; transecting a subcutaneous layer of the patient's skin, opening the anterior rectus sheath, opening the posterior rectus sheath and peritoneum, and a trocar advanced into the abdomen, which is insufflating with CO₂, advancing a second trocar through the anterior and posterior rectus sheaths at the planned exit, advancing the trocar into the peritoneum under direct vision, inserting the tunneling sheath via the peri-umbilical with the tip of the sheath located within the peritoneum and with a cuff located in the rectus muscle, forming a subcutaneous tunnel, placing a cuff in the subcutaneous tunnel, advancing an internal peritoneal dialysis catheter through the tunneled sheath and into the Pouch of Douglas using direct visualization, cutting the internal peritoneal dialysis catheter to length, securing the catheter to the tunneled sheath, testing the catheter, desufflating the abdomen while leaving camera in place to confirm catheter remains in the appropriate location, removing the trocar, and closing the rectus sheath and wounds closed using resorbable sutures.

### Methods of Performing Peritoneal Dialysis

In some embodiments, continual peritoneal dialysis is performed by advancing a first peritoneal dialysis catheter through the long-term use sheath and performing peritoneal dialysis with the first peritoneal dialysis catheter.

In some embodiments, the first peritoneal dialysis catheter is exchanged with a second peritoneal dialysis catheter by removing the first peritoneal catheter from the long-term use sheath and advancing the second peritoneal catheter through the long-term use sheath. In some embodiments, the method further comprises performing peritoneal dialysis through the second peritoneal catheter advanced through the long-term use sheath. In some embodiments, continual peritoneal dialysis involves the use of 3, 4, 5, 6, 7, 8, 9, 10 or more peritoneal dialysis catheters.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-31. (canceled)

32. A long-term use sheath for facilitating peritoneal dialysis, the long-term use sheath comprising:
a sheath body having an inner lumen, wherein the lumen is configured to accommodate advancement of a first peritoneal dialysis catheter therethrough and exchange of the first peritoneal dialysis with a second peritoneal dialysis catheter;
a first cuff coupled to the sheath body and adapted to secure to a rectus muscle of the subject; and
a second cuff coupled to the sheath body and adapted to secure to skin of the subject.

33. The long-term use sheath of claim 32, wherein at least a portion of the long-term use sheath is impregnated with one or more of an anti-microbial agent, an anti-bacterial agent, copper, or silver.

34. The long-term use sheath of claim 32, wherein at least a portion of the long-term use sheath is pliable.

35. The long-term use sheath of claim 32, wherein at least a portion of the long-term use sheath is rigid.

36. The long-term use sheath of claim 32, wherein at least a portion of the long-term use sheath has a rigid bend.

37-39. (canceled)
